(19) United States
(12) Patent Application Publication
(10) Pub. No.: US 2024/0365800 A1
(43) Pub. Date: Nov. 7, 2024

(54) SOLID SOLUBLE COMPOUND FOR APPLICATION AS A CARRIER OF ACTIVE AGENTS OR MULTIFUNCTIONAL COATING ON PLANT AND FRUIT STRUCTURES AND METHOD OF OBTAINMENT THEREOF

(71) Applicant: UNIVERSIDAD DE LOS ANDES, Bogotá (CO)

(72) Inventors: Andres David ROJAS VERA, Bogotá (CO); Ariel Mauricio VACA BOHÓRQUEZ, Bogotá (CO); Felipe SALCEDO GALÁN, Bogotá (CO)

(21) Appl. No.: 18/686,161

(22) PCT Filed: Aug. 23, 2021
(86) PCT No.: PCT/IB2021/057732

§ 371 Date: Feb. 23, 2024

(51) Int. Cl. A23B 7/154; A23B 7/16; A23L 3/349; A23L 3/3508; A23L 3/3517; A23L 3/3544; A23L 3/3562; A61K 31/202; A61K 31/593; A61K 31/733; A61K 33/00; A61K 33/06; A61K 33/26; A61K 35/00; A61K 35/747; A61K 45/06

(52) U.S. Cl. CPC A23B 7/154; A23B 7/16; A23L 3/349; A23L 3/3508; A23L 3/3517; A23L 3/3544; A23L 3/3562; A61K 31/202; A61K 31/593; A61K 31/733; A61K 33/00; A61K 33/06; A61K 33/26; A61K 35/747; A61K 45/06; A61K 2035/115

(57) ABSTRACT

The present invention relates to a solid soluble compound for application as a carrier of active agents or multifunctional coating on plant and fruit structures and method of obtaining thereof comprising a polysaccharide which is modified in situ during the obtaining of said composite material (A) together with a plasticizing agent and a surfactant agent wherein the polysaccharide is in a ratio of 20% to 40% w/w, the plasticizing agent is in a ratio of 2% to 23% w/w, and the surfactant agent is in a ratio of 2% to 23% w/w. The invention also relates to the multifunctional coating (B) comprising a composite material (A) and a solvent, and wherein both the composite material (A) and the multifunctional coating (B) may have additional active agents and nutritional agents. The invention relates to the method of preparation of the composite material (A), wherein the chemical modification of the polysaccharide is performed in situ using a chemical modifying agent. Furthermore, the invention relates to a method for the obtainment of the the multifunctional coating by dispersion of the composite material (A) in a solvent and to a method of depositing the multifunctional coating on food products such as plants and fruits.

### TECHNICAL FIELD OF THE INVENTION

The present technology relates to the field of green and environmentally friendly technologies, since it provides a composite material that protects the surfaces and structures of plants and fruits from oxidation, loss of water and volatile components of interest, and pathogens, thus providing a protective barrier for plants and fruits, both for their transportation and shelf life.

### BACKGROUND OF THE INVENTION

The proliferation of disease-causing microbial pathogens on the surface of foods, particularly plants and fruits, is a latent problem today, given that pathogens also cause destruction of plant tissues and damage inside them, leading to cell death of plant structures with the consequent loss of these foods. In the case of fruits, inadequate management of these pathogens leads to product losses during harvest and post-harvest when fruits or any other plant is subjected to prolonged storage and transportation times, which promotes premature ripening and the occurrence of diseases caused, for example, by fungi in the supply chain.

In the field of patents, international patent WO2015/134865 provides biopolymer-based edible coatings suitable for coating perishable products, such as food items, wherein the coating materials are protein-based. The coatings prepared in accordance with this patent comprise an amphiphilic polypeptide having an overall hydrophobicity of 65%, such that at least 65% of the amino acid residues of the amphiphilic polypeptide are hydrophobic amino acid residues and wherein the coating comprises an amorphous fraction and a crystalline fraction. Proteins may be fibroins, actins, collagens, catenins, claudins, coilins, elastins, elaunins, extensins, fibrillins, keratins, tubulins, viral structural proteins, zein proteins (seed storage proteins) and any combination thereof.

In turn, patent CN104073000 describes an edible fluid sericin maintenance film comprising 10 to 15 parts of sericin protein fluid and 85 to 90 parts of a matrix, wherein the matrix comprises 5 to 8 parts of polyvinyl alcohol, 1 to 2 parts of glycerol, 0.6 to 0.9 parts of sodium benzoate, 1 to 2 parts of carboxymethylcellulose, 1 to 2 parts of sodium alginate, and 85 to 90 parts of water. The film forms a thin layer of fluid coating on fruits and plants to enhance the protective effect of food skin layers, reduce mechanical damage, control water loss, prevent shrinkage, wilting, and spoilage, as well as maintain product flavor.

On the other hand, Patent MX2011013627 describes a process to manufacture an edible coating based on candelilla wax, jojoba oil, gum arabic, ellagic acid, and water, which are mixed to obtain an emulsion of the components, wherein gum arabic is soluble in water and acts as a dispersing agent. Jojoba oil is intended to act as a plasticizer, thus providing the permeability to the final coating and wherein the pH must be adjusted to 9, as this is the optimum pH to homogenize the components; temperature is also raised to 80° C. to prevent the wax from being solid and homogenize it at 2800 rpm until a perfect homogenization is obtained and the addition of ellagic acid as an active component that improves the functionality of the coating.

Patent US 2003203084 teaches a coating for food products, wherein a virgin chitosan polymer is added to a solution of acid and water in an amount sufficient to form an edible composition having a solids content greater than 5% and a liquid viscosity. The composition is applied to food products, such as fruits, plants, and nuts, to provide an edible protective coating for the food products, wherein the chitosan can be pre-hydrolyzed to a lower molecular weight so that a gel does not form when the partially hydrolyzed chitosan is mixed with the acid water solution. The coating also comprises a preservative such as sodium benzoate and/or an adhesion additive such as zinc acetate, together with a wetting agent, and/or one or more additives from the group consisting of virgin and/or modified carbohydrates, proteins, hydrocolloids, lipids, oils, gums, and waxes, natural and/or synthetic, which may be added to the composition before it is applied to the food product.

Thus, it is clear that a problem remains with protecting surfaces of plant and fruit structures under high oxygen concentration conditions which lead to their degradation, loss of water and volatile components of interest, as well as the proliferation of microbial pathogens that cause damage to plants and fruits. Therefore, the present invention provides a solution to these and other problems related to the preservation of food, especially plants and fruits, using a composite material which, when placed in a dispersion, allows reducing the losses of plant products during harvest and post-harvest, also enabling the reduction of logistics costs and problems associated with damage to the products when subjected to long storage and transport periods, improving the shelf life of fruits and other plants since it helps to maintain their integrity for a longer time, which leads to increasing and/or maintaining their quality over time. Additionally, the composite material and its suspension according to the present invention creates passive and active protection barrier against pathogens, which can promote the concentration of nutrients that strengthen plant response to such pathogens.

### DESCRIPTION OF THE DRAWINGS

FIG. 1 is a schematic representation of the invention, which is composed of a solid soluble compound (A), a multifunctional coating (B) and its production method.

FIG. 2 presents an infrared spectrum that shows the reduction of hydrogen bonds (lipophobicity) of the test polysaccharide (starch) modified in the process of obtaining the composite material (A) compared to unmodified starch.

FIG. 3 presents an infrared spectrum that shows the esterification of the test polysaccharide (starch) modified in the process of obtaining the composite material (A) compared to unmodified starch.

FIG. 4 presents an infrared spectrum that shows the hydrolysis of the test polysaccharide (starch) modified in the process of obtaining the composite material (A) compared to unmodified starch.

FIG. 5 presents the vapor permeability behavior of films according to the invention due to the presence of the modified polysaccharide (modified starch).

FIG. 6 presents the increase in shelf life of fruit coated with the multifunctional coating according to the present invention as compared to uncoated fruit.

FIG. 7 presents the reduction in weight loss of fruit with the coating according to the present invention compared to uncoated fruit.

FIG. 8 presents the delay in color change and reduction of effects due to anthracnose of mango fruit over time for fruit with multifunctional coating according to the present invention compared to uncoated fruit (control).

FIG. 9 presents the delay in color change of common mango fruit with multifunctional coating over time compared to uncoated fruit (control).

FIG. 10 presents the delay in color change of Hass avocados when treated with the multifunctional coating according to the present invention compared to untreated ones.

FIG. 11 presents the preservation of Hass avocados treated with the multifunctional coating against surface fungal action compared to untreated Hass avocados.

FIG. 12 presents the preservation of Hass avocados treated with the multifunctional coating against internal fungal action compared to untreated Hass avocados.

### SUMMARY OF THE INVENTION

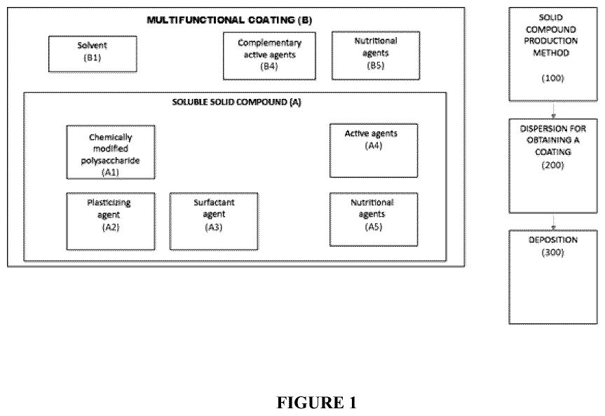

The present invention relates to a solid soluble compound (A) comprising a polysaccharide which is modified in situ during the obtaining of said composite material (A) together with a plasticizing agent and a surfactant agent wherein the polysaccharide is in a ratio of 20% to 40% w/w, the plasticizing agent is in a ratio of 2% to 23% w/w, and the surfactant agent is in a ratio of 2% to 23% w/w. The invention also relates to the multifunctional coating (B) comprising a composite material (A) and a solvent, and wherein both the composite material (A) and the multifunctional coating (B) may have additional active agents and nutritional agents. The invention relates to the method of preparation of the composite material (A), wherein the chemical modification of the polysaccharide is performed in situ using a chemical modifying agent. Furthermore, the invention relates to a method for the obtainment of the multifunctional coating by dispersion of the composite material (A) in a solvent and to a method of depositing the multifunctional coating on food products such as plants and fruits (FIG. 1).

### DETAILED DESCRIPTION OF THE INVENTION

In a first aspect, the invention relates to a composite material (A) for a carrier product of active and nutritional agents of interest that can be used as a multifunctional coating for plant structures comprising a polysaccharide chemically modified in situ, a plasticizing agent, and a surfactant, wherein the polysaccharide is in a ratio of 20% to 40% w/w, the plasticizing agent is in a ratio of 2% to 23% w/w, and the surfactant is in a ratio of 2% to 23% w/w.

The composite material (A) according to the present invention has a humidity of between 0% and 15% and may be in the form of sheets with a size between 100 and 500 micrometers. Moreover, the composite material (A) may be in the form of granules with a size between 100 and 500 micrometers.

According to the present invention, the polysaccharide that is chemically modified in situ is selected from the group consisting of macromolecules composed of hexose and pentose carbohydrate monomers, such as galactose, glucose, arabinose, xylose, and ribose, and combinations of said monomers.

According to the present invention, the chemical modification carried out during the obtaining of the composite material (A) is performed by crosslinking, esterification, and hydrolysis of said polysaccharide.

According to the present invention, the polysaccharide that is chemically modified in situ is selected from the group consisting of macromolecules composed of hexose and pentose carbohydrate monomers, such as galactose, glucose, arabinose, xylose and ribose, starches, celluloses, and other heteropolysaccharides, all in food grade and commercially available, wherein the chemical modification carried out during the obtaining of the composite material (A) is made by crosslinking, esterification, and hydrolysis of said polysaccharide.

The modification of the polysaccharide consists of esterification, hydrolysis, and crosslinking performed sequentially in situ during the obtaining of the composite material (A) according to the invention. Preferably, all of them are carried out with the same chemical modifying agent. The modifying agent may be selected from the set of polycarboxylic acids having more than three carboxylic groups, e.g., EDTA, trifunctional fatty acids, malic acid, succinic acid, propane-1,2,3-tricarboxylic acid, citric acid. The chemical modifying agent used in the in situ modification of the polysaccharide of the composite material (A) may be selected from the group consisting of citric acid, isocitric acid, aconitic acid, propan-1,2,3-tricarboxylic acid, and trimesic acid, all in food grade. Preferably, the chemical modifying agent is citric acid.

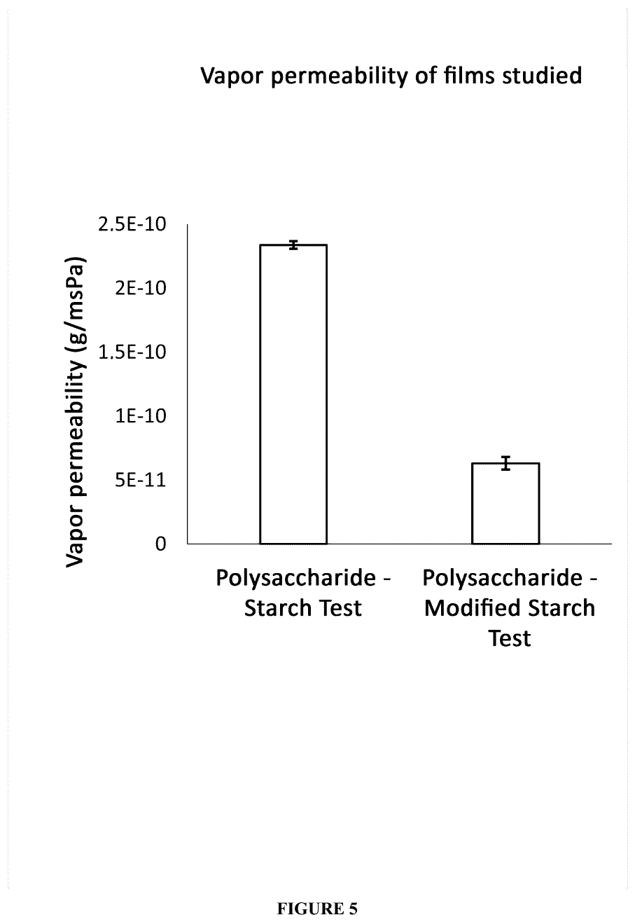

This in situ modification reaction of the polysaccharide using the chemical modifying agent allows the polysaccharide to create a chemical network, reduce lipophobicity to promote the ability of the matrix to accept active compounds of both hydrophilic and lipophilic nature, more easily absorb the solvent of the composite material (A) to obtain the multifunctional coating (B) in dispersion, improve the mechanical capacity of the matrix, and reduce water vapor permeability as illustrated in FIG. 5, in which it is evident that the participation of the polysaccharide modified in situ significantly reduces water vapor permeability in the films formed.

In some compositions for the composite material (A), the chemical modifying agent may also have a plasticizing agent functionality since it provides additional plastic properties to the in situ modified polysaccharide matrix, further conferring ductility and increased resistance to permanent mechanical deformation, and giving hygroscopicity to the composite material (A).

The plasticizing agent of the composite material (A) according to the present invention may be selected from polyols (polyalcohols), surfactants, polycarboxylic acids, and water, wherein the polyols may be, but are not limited to, glycerol/glycerol, propylene glycol, and sorbitol, all in food grade.

Thus, the person skilled in the art will understand, in light of on the present specification, that the plasticizing agent may be any food grade non-toxic low molecular weight polar or amphiphilic molecule which may be in the group of polyols (polyalcohols), surfactants, poly carboxylic acids, and water. Polyols, which are polyhydric alcohols, may be selected from glycerol/glycerine, propylene glycol, sorbitol.

The preferred plasticizing agent for the composite material (A) according to the present invention is glycerin because it is a small molecule and is easily available, since it can be the byproduct of various industrial processes.

The surfactant agent of the composite material (A) according to the present invention is by definition a food grade non-toxic amphiphilic, hydrophilic surfactant molecule which may be selected from polysorbates, proteins, sucrose esters of fatty acid, and monoacylglycerides. In a preferred embodiment of the invention, the surfactant agent is polyoxyethylene (20) sorbitan monolaurate (Tween 20). The action of the surfactant agent within the composite material (A) is to disperse the composite material (A) when it comes into contact with the solvent, allowing any agglomerations of the composite material (A) that may occur to be destructured at the macroscopic level. Additionally, the surfactant agent helps to bind non-polar elements of the composite (A) and provides stability to the dispersion of the composite (A) in the solvent at room temperatures (12° C. to 30° C.).

In another preferred embodiment of the invention, the composite material (A) further comprises active agents which are between 0% and 12%, wherein the active agents are selected from oleic acid, linoleic acid, sucrose monolaurate, sucrose monodecanoate, glyceryl monostearate, tannic acid, ascorbic acid, gallic acid, ellagic acid, anthocyanins, hexanal, hexanol, linseed oil, and essential oils of thyme and citronella, biopolymers such as chitosan, cellulose nanoparticles and xanthan gum, salts or ions such as CaCl₂, NaCl, and Fe (III) ions, enzymes and proteins of microbiological origin, as well as microorganisms or extracts thereof, all in food grade. Additionally, active agents with antifungal activity such as thiabendazole, peracetic acid, or others allowed for post-harvest use in fruits with inedible peel with concentrations lower than permitted MRLs (maximum pesticide residue limits), as well as components with aseptic activity against viruses, such as hydrogen peroxide, glutaraldehyde, peracetic acid, acetic acid, and salicylic acid, compounds of very low toxicity with concentrations lower than permitted MRLs.

These active agents may be bioactive agents of plant origin, of biotechnological production, or molecules of chemical synthesis that react or modify the metabolism of plant structures that come into contact with the composite material (A) when it is dispersed in a solvent or mixture of solvents and can provide antimicrobial (antibacterial, antifungal, and antiviral), antioxidant, and hydrophobic activity and can also provide nutrients for plants when applied in the pre-harvest and harvest period. Likewise, active agents such as biological control microorganisms are known for their antimicrobial activity commonly and are used in pre-harvest, harvest, and post-harvest with very low or no toxicity.

In another preferred embodiment of the invention, the composite material (A) may further comprise nutritional agents present between 0% and 12%, which are selected from the group consisting of vitamins such as B-complex vitamins and vitamin D, fatty acids such as omega 3 and omega 6, iron complexes, potassium, magnesium, inulin, Lactobacillus, and salts, all in food grade and commercially available.

In turn, nutritional agents are nutraceutical compounds that provide added value to the nutritional content of the composite material (A), to the products derived from said composite material (A), and to the fruit and plant products that said products may coat.

According to the above, it has been found that the components of the composite material (A), mainly the in situ modified polysaccharide and the surfactant agent act synergistically to disperse the composite material (A) in water or other solvents at room temperature without requiring significant temperature increases or pH modifications of the medium in which they are dispersed. Furthermore, it has been found that the in situ chemically modified polysaccharide acts as a matrix with a lamellar microstructure or porous granules that provides both physical and chemical anchoring spaces for the transport of active compounds at different levels (molecular and microscopic) thanks to its structure and its capacity to absorb both aqueous and lipidic liquids.

In a second aspect, the present invention refers to a multifunctional coating (B) in dispersion for coating plant and fruit structures comprising a composite material (A) for a multifunctional coating product for plant structures comprising a polysaccharide chemically modified in situ, a plasticizing agent, a surfactant, wherein the composite material is dispersed in a solvent and the polysaccharide is in a ratio of 20% to 40% w/w, the plasticizing agent is in a ratio of 2% to 23% w/w, and the surfactant is in a ratio of 2% to 23% w/w. Moreover, the multifunctional coating (B) in dispersion may further comprise complementary active agents which are between 0% and 12% and complementary nutritional agents which are between 0% and 12%.

Thus, the complementary active agents in the multifunctional coating (B) in dispersion according to the present invention are in a ratio of 0 mg/ml to 1 mg/ml and are selected from oleic acid, linoleic acid, sucrose monolaurate, sucrose monodecanoate, glyceryl monostearate, tannic acid, anthocyanins, hexanal, hexanol, linseed oil, essential oils of thyme and citronella, ascorbic acid, gallic acid, CaCl₂, NaCl, Fe (III) ions, enzymes and proteins of microbiological origin, as well as microorganisms or extracts thereof, all in food grade. Additionally, disinfectant solutions for fruits and plants. Active agents with antifungal activity such as thiabendazole, peracetic acid, or others allowed for post-harvest use in fruits with inedible peel with concentrations lower than permitted MRLs, as well as components with aseptic activity against viruses, such as hydrogen peroxide, glutaraldehyde, peracetic acid, acetic acid, and salicylic acid, compounds of very low toxicity with concentrations lower than permitted MRLs.

Complementary nutritional agents in the multifunctional coating (B) in dispersion according to the present invention are in a ratio of 0 mg/ml to 1 mg/ml and are selected from the group consisting of B-complex vitamins, vitamin D, omega 6, iron complexes, potassium, magnesium, inulin, Lactobacillus, and edible salts.

In one embodiment of the present invention, the composite material (A) is in a concentration or ratio of 0.5 mg/ml to 50 mg/ml.

In one embodiment of the present invention, the solvent for the multifunctional coating (B) according to the present invention is a solution of aqueous solvents or water-in-oil emulsions, wherein the solvent is selected from the group consisting of alcohol, oils, surfactants, and mixtures thereof and wherein the solvent contains between 80% and 100% water and the complement is the mixture of alcohol, oils, and surfactants.

This multifunctional coating (B) is a suspension that adheres to food product surfaces, e.g., plants and fruits, and then upon drying on such surfaces forms a solid film. This multifunctional coating (B) is usually a translucent white suspension solution with viscosities between 0.004 Pa·s and 0.01 Pa·s that exhibits surface adhesion characteristics due to its surface tension, forming a mechanically resistant film that does not disintegrate over time and with a higher resistance to abrasion. In addition, thanks to its translucency, after drying on the surface of the food product, it does not modify the appearance of the plant or fruit structure, thus providing greater confidence to end consumers.

Moreover, due to the viscosity of the multifunctional coating (B) in suspension, it can be applied by any known depositing mechanism on plant surfaces without damaging the application or storage infrastructure of the multifunctional coating (B) and retaining its food safety properties to be consumed directly by consumers or easily removed by washing under running water.

In a third aspect, the invention relates to a method for the preparation or manufacture of the composite material (A) for multifunctional coating product for plant and fruit structures characterized in that it comprises the following steps:
a) Preparing a solution of plasticizing agent in water, wherein the plasticizing agent is at a concentration of 5% to 25% w/w with respect to the polysaccharide in solid state, wherein the solution is stirred at a speed between 300 and 500 rpm at a temperature between 18° C. and 30° C.;
b) Adding to the solution prepared in step a) a chemical modifying agent at a concentration of 5% to 30% w/w with respect to the polysaccharide, maintaining the stirring between 300 and 500 rpm and the temperature between 25° C. and 30° C.;
c) Adding to the solution prepared in step b) the polysaccharide at a mass aggregation rate of 5 to 10 g/s until reaching a concentration between 1% to 10% w/v and raising the temperature between 30° C. and 50° C.;
d) Solubilizing and hydrolyzing the polysaccharide in situ by increasing the temperature at a rate of 0.5 to 5° C./min until reaching a temperature between 80° C. and 90° C.;
e) Maintaining the final temperature and stirring constant between 300 and 500 rpm;
f) Condensing by reducing the temperature at a constant rate until reaching room temperature (18° C.-30° C.); and
g) Drying the product obtained in step f) by spray-drying with drying air at a temperature of 110° C.-200° C. and a flow rate of 100-1000 L/h or forced convection with reduced humidity of warm air at room temperature (18° C.-60° C.).

According to the method for the preparation or manufacture of the composite material (A) above, in step b) the chemical modifying agent is selected from the group consisting of citric acid, isocitric acid, aconitic acid, propan-1,2,3-tricarboxylic acid, and trimesic acid, all in food grade with a concentration between 5% and 30% by weight with respect to the polysaccharide being modified in situ. In a preferred embodiment, the chemical modifying agent is food grade citric acid.

In one embodiment of the invention, the method for the preparation or manufacture of the composite material (A) may have an additional step before or after step f), wherein said additional step consists of adding a surfactant by permanent drip at a temperature between 18° C. and 80° C. with stirring between 500 and 1000 rpm, wherein the surfactant is selected from the group of polysorbates, proteins, sucrose esters of fatty acid, and monoacylglycerides. In a preferred embodiment of the invention, the surfactant agent is polyoxyethylene (20) sorbitan monolaurate (Tween 20).

In one embodiment of the invention, the method for the preparation or manufacture of the composite material (A) comprises a step after final step g), wherein particle size reduction is carried out by grinding, for example, in ball mill or any other conventional mill, by breaking or cutting the dry flakes of the composite material (A) until reaching a particle size between 100 and 500 micrometers.

In one embodiment of the invention, the method for the preparation or manufacture of the composite material (A), the active agents, and the nutritional agents may be added in any order and steps, except for step c).

The polysaccharide used in the method of preparation or manufacture of the composite material (A) may be selected from hexose and pentose carbohydrate monomers, such as galactose, glucose, arabinose, xylose, and ribose, among others.

In one embodiment of the invention, in the method for the preparation or manufacture of a composite material (A), the plasticizing agent is selected from polyols (polyalcohols), surfactants, polycarboxylic acids, and water. In a preferred embodiment of the invention, in the method the plasticizing agent is selected from polyols of the group consisting of glycerol/glycerol, propylene glycol, sorbitol, all in food grade, and most preferably the plasticizing agent is glycerol (glycerin).

In one embodiment of the invention, in the method for the preparation or manufacture of a composite material (A), the surfactant is selected from polysorbates, proteins, sucrose esters of fatty acid, and monoacylglycerides. In a preferred embodiment, the surfactant agent is polyoxyethylene (20) sorbitan monolaurate (Tween 20).

In one embodiment of the invention, in the method for the preparation or manufacture of a composite material (A), the active agents are selected from oleic acid, linoleic acid, sucrose monolaurate, sucrose monodecanoate, glyceryl monostearate, tannic acid, gallic acid, ellagic acid, ascorbic acid, anthocyanins, hexanal, hexanol, linseed oil, and essential oils of thyme and citronella, biopolymers such as chitosan, xanthan gum, and cellulose nanoparticles, salts or ions such as CaCl₂, NaCl, Fe (III) ions, as well as microorganisms, all in food grade. Additionally, disinfectant solutions for fruits and plants. Components with antifungal activity such as thiabendazole, peracetic acid, or others allowed for post-harvest use in fruits with inedible peel with concentrations lower than permitted MRLs, as well as components with aseptic activity against viruses, such as hydrogen peroxide, glutaraldehyde, peracetic acid, acetic acid, and salicylic acid, compounds of very low toxicity with concentrations lower than permitted MRLs.

In one embodiment of the invention, in the method for the preparation or manufacture of a composite material (A), the nutritional agents are selected from the group consisting of vitamins such as B-complex vitamins and vitamin D, fatty acids such as omega 3 and omega 6, iron complexes, potassium, magnesium, inulin, Lactobacillus, and salts.

In a fourth aspect, the invention relates to a method for the obtainment of the multifunctional coating in dispersion from the composite material (A) in a solvent, wherein the method comprises the steps of:
i. Forming a paste of the solid composition (A) by wetting and machining by adding water or solvent in a ratio of solid composition (A): solvent or water 1:1 to 5:1;
ii. Adding the paste obtained in step i) to the solvent or water up to a concentration of solid composition (A) between 0.5 g to 50 g per liter of solvent and stirring until obtaining a homogeneous solution;
iii. Adding complementary active and nutritional agents.

In one embodiment of the invention, in the method for the obtainment of the multifunctional coating in dispersion, the additional step of adding active and nutritional agents may be performed before, during, or after step ii).

In a fifth aspect, the invention also relates to the method of depositing a multifunctional coating in dispersion for plant structures from a solid composition (A), comprising the steps of:
A. Contacting the soluble coating solution with the surface of the plant or fruit;
B. Drying the plant or fruit.

In a preferred embodiment of the invention, in the method of depositing a multifunctional coating for plant structures from a composite material (A), stage A is selected from solution dipping, brushing, spraying, or bathing.

### EXAMPLE

Composite material A was obtained by mixing industrial grade glycerin in water in a ratio of 0.5% w/v to water at a room temperature of 18° C. and at 300 rpm. After 10 minutes, industrial grade citric acid was added until reaching a concentration in water of 0.6% w/v. Temperature and stirring conditions were maintained. After another 10 minutes, a polysaccharide (with less than 10% moisture) was added at a rate of 10 g/s until reaching a 2% w/v concentration with respect to water. Temperature was increased up to 30° C. and stirring was increased up to 500 rpm. After 10 minutes, a heating ramp was started at a rate of 2° C./min until reaching a temperature close to 90° C. Constant stirring and the final temperature (90° C.) was maintained for an additional 30 minutes after reaching this temperature. Then, the heating was stopped, and the solution was left to cool down to room temperature (18° C.). Subsequently, 0.05% w/v tannic acid was added and mixed for 10 minutes at 1000 rpm. Polyoxyethylene sorbitan monolaurate (Tween 20) was added to the solution by dripping at 1000 rpm for 10 minutes until reaching a concentration of 0.3% w/v. Linseed oil was added to the solution by dripping at 1000 rpm for 10 minutes until reaching a concentration of 0.1% w/v. Then, the obtained solution was dried on a metal tray in a food dryer with a continuous flow of dry air (20% RH) at a temperature of 25° C. After drying, the film formed was removed, cut, ground in a knife mill and then in a ball mill.

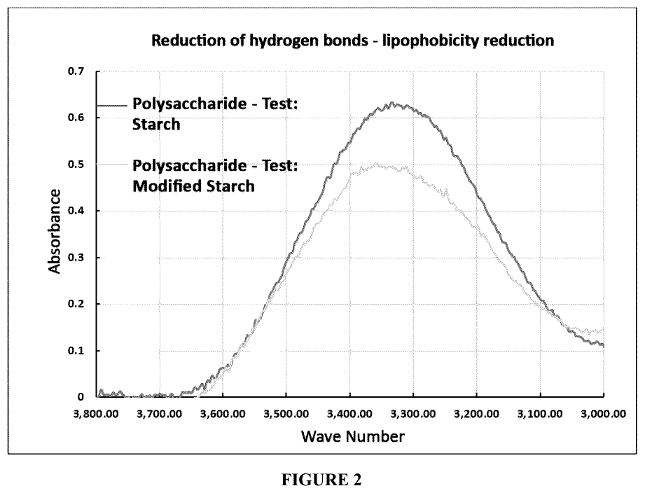

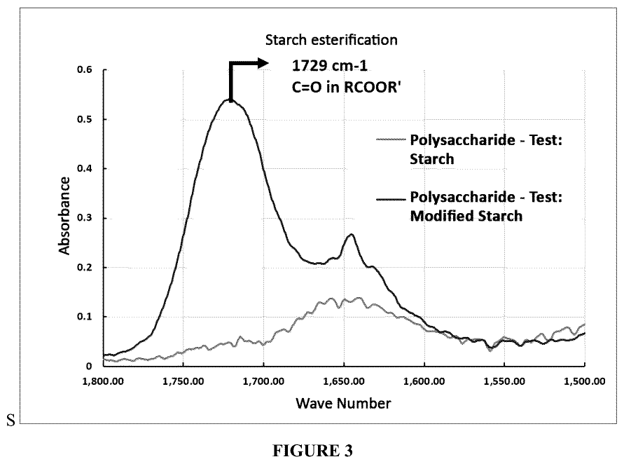

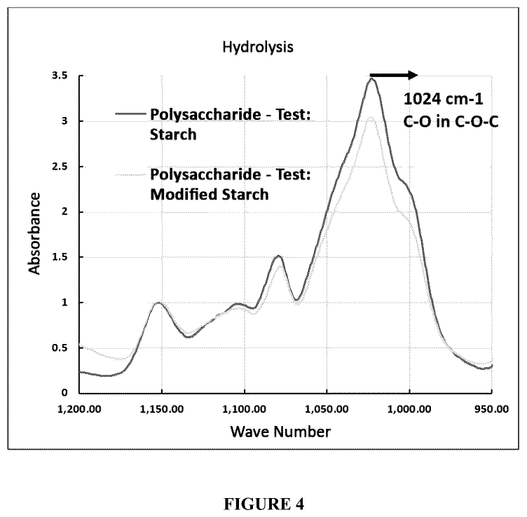

During the process of obtaining the composite material (A), a sample was collected to verify that the modification was being made in situ. As can be seen in FIGS. 2, 3 and 4, corresponding to infrared spectra, the behavior of starch in the sample compared to the behavior of unmodified starch shows a decrease in hydrogen bonding for modified starch (FIG. 2) and an absorption peak at 1,729 cm⁻¹ for the C═O bond product of esterification in R—COOR′ (FIG. 3) and hydrolysis by reduction of the peak in modified starch at 1,024 cm⁻¹ for the C—O bond in the C—O—C group.

The dry and ground compound A was moistened with little water and macerated until obtaining a homogeneous paste. Then, the paste formed was mixed with water until obtaining a concentration considering the dry weight of compound A of 50 g per 100 ml, and mixed at 1000 rpm. Next, the solution was diluted in 900 ml of additional water. This resulted in the multifunctional coating solution (B).

The multifunctional coating solution (B) was sprayed by means of a manual spray atomizer on Hass avocados, wetting them completely and then leaving them to dry at room temperature for 20 minutes. This formed the functional solid coating on the avocado epidermis, which is imperceptible to touch and sight.

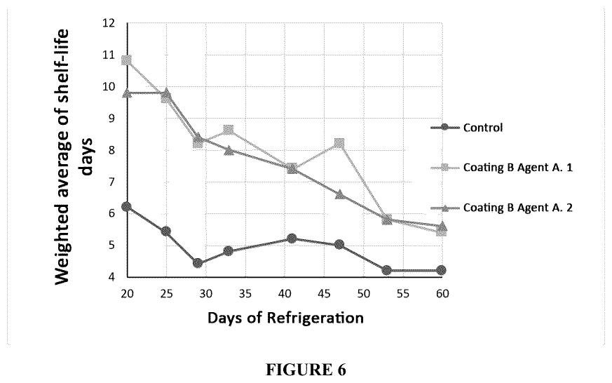

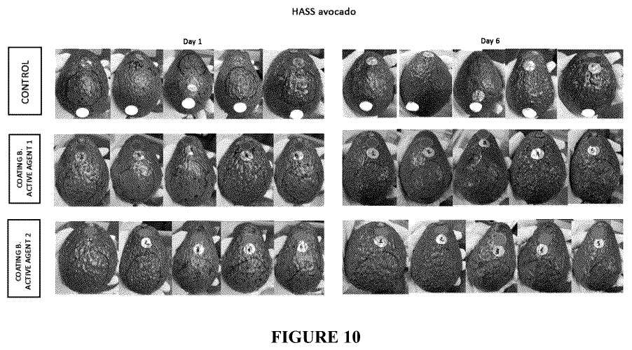

Hass avocados coated with the multifunctional coating (B) increased their shelf life by up to 6 days, as shown in FIG. 6, which corresponds to the weighted average of shelf life days versus refrigeration days. These Hass avocados without (control) and with multifunctional coating (B) were subjected to cold chain storage at 5-7° C. and 70-90% relative humidity (RH) in natural ambient atmosphere for 20 days. After the cold chain was completed, uncoated avocados ripened after 4 and 6 days. Avocados with multifunctional coating (B) ripened within 10-11 days with no change in flavor and appearance compared to the control. The conditions of the various samples are shown in FIG. 10.

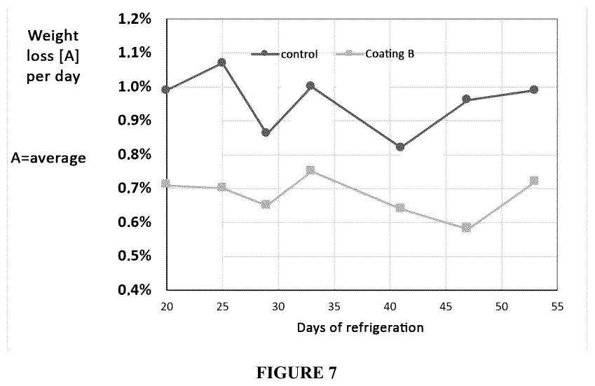

In addition, a study was carried out to examine the percentage of weight loss of the samples, which is related to the loss of water by evaporation and the loss of volatile substances from the products. This study found that, during refrigeration time, the average loss is significantly reduced for the products coated with the multifunctional coating (B) compared to the control (see FIG. 7).

Furthermore, it was observed that Hass avocados with multifunctional coating (B) stored at ambient conditions and with cold chain showed very low or no signs and symptoms of fungal proliferation (stem end rot and body rot).

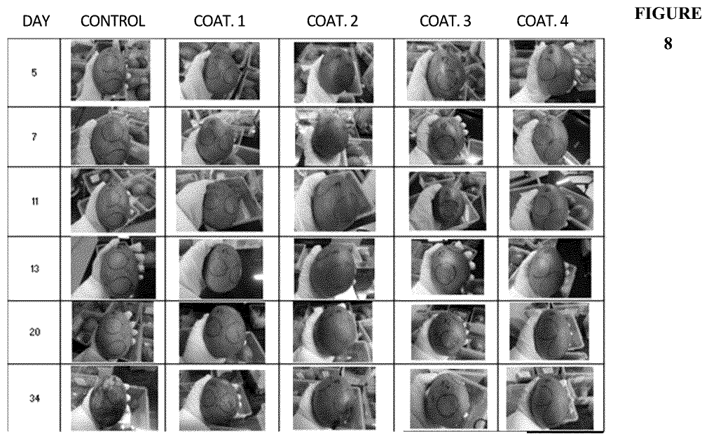

A study was also conducted on a different fruit, mango. FIG. 8 shows the behavior of the control fruit, the fruit with conventional coatings (coating 1, 2 and 3), and the fruit with coating 4 (multifunctional coating (B)) under the same storage conditions. As can be observed in the photographs, the fruit with the coating according to the present invention maintains its appearance from day 5 to day 34 and its ripening is delayed in comparison to the control fruit and other conventional coatings.

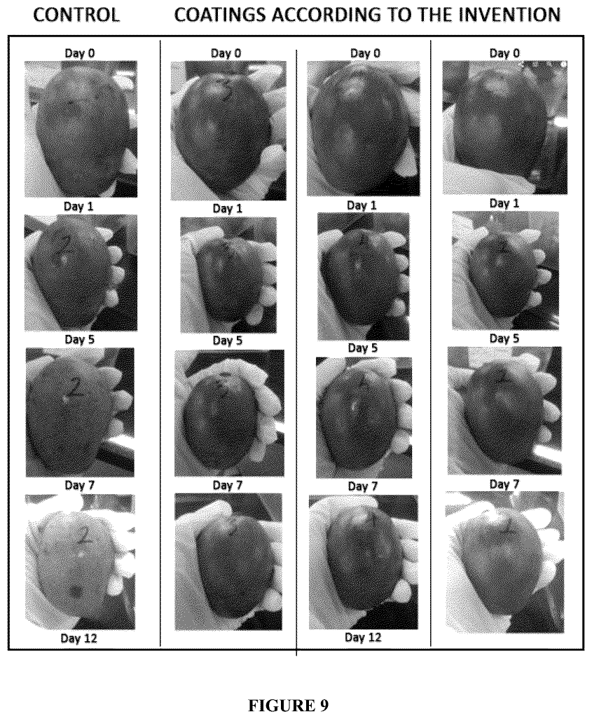

FIG. 9 illustrates common mango fruit preservation for coatings according to the present invention as compared to control fruit under the same storage conditions.

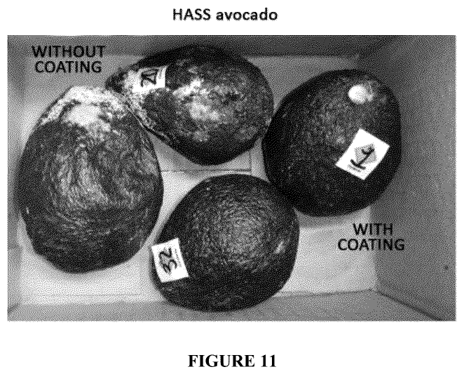

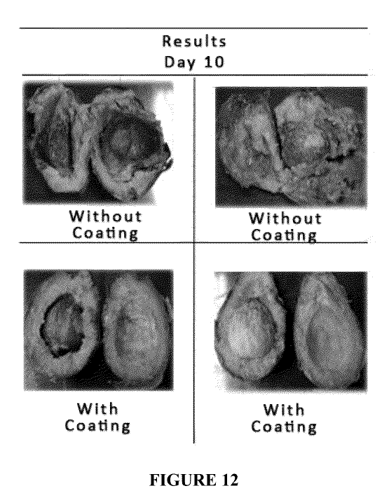

Fungal activity is also evident in other fruits such as Hass avocado, as shown in FIG. 11, which shows the preservation of coated fruits after a period of time (10 days) in storage compared to uncoated fruits. FIG. 12 also shows how the internal integrity of the fruit is benefited.

1. A composite material (A) for a carrier product for active and nutritional agents of interest that can be used as a multifunctional coating for plant structures characterized in that it comprises a polysaccharide chemically modified in situ, a plasticizing agent, and a surfactant agent.

2. The composite material (A) for a multifunctional coating product for plant structures according to claim 1, characterized in that the polysaccharide is in a ratio of 20% to 40% w/w, the plasticizing agent is in a ratio of 2 to 23% w/w, and the surfactant agent is in a ratio of 2 to 23% w/w.

3. The composite material (A) for a multifunctional coating product for plant structures according to claim 1, characterized in that it has a relative humidity of between 0% and 15%.

4. The composite material (A) for a multifunctional coating product for plant structures according to claim 1, characterized in that it is in the form of sheets with a size between 100 and 500 micrometers.

5. The composite material (A) for a multifunctional coating product for plant structures according to claim 1, characterized in that it is in the form of granules with a size between 100 and 500 micrometers.

6. The composite material (A) for a multifunctional coating product for plant structures according to claim 1, characterized in that the polysaccharide which is chemically modified in situ is selected from the group consisting of macromolecules composed of hexose and pentose carbohydrate monomers, such as galactose, glucose, arabinose, xylose, and ribose.

7. The composite material (A) for a multifunctional coating product for plant structures according to claim 1, characterized in that the chemical modification is provided by crosslinking, esterification, and hydrolysis.

8. The composite material (A) for a multifunctional coating product for plant structures according to claim 1, characterized in that the plasticizing agent is selected from polyols (polyalcohols), surfactants, polycarboxylic acids, and water.

9. The composite material (A) for a multifunctional coating product for plant structures according to claim 9, characterized in that the polyols are selected from the group consisting of glycerol/glycerin, propylene glycol, and sorbitol, all in food grade.

10. The composite material (A) for a multifunctional coating product for plant structures for multifunctional coating product for plant structures according to claim 1, characterized in that the surfactant agent is selected from polysorbates, proteins, sucrose esters of fatty acid, and monoacylglycerides.

11. The composite material (A) for a multifunctional coating product for plant structures according to claim 9, characterized in that the surfactant agent is polyoxyethylene (20) sorbitan monolaurate (Tween 20).

12. The composite material (A) for a multifunctional coating product for plant structures according to claim 1, characterized in that it further comprises active agents and nutritional agents, wherein the active agents are present between 0% and 12%, and the nutritional agents are present between 0% and 12%.

13. The composite material (A) for a multifunctional coating product for plant structures according to claim 13, characterized in that the active agents are selected from oleic acid, linoleic acid, sucrose monolaurate, sucrose monodecanoate, glyceryl monostearate, tannic acid, gallic acid, ellagic acid, ascorbic acid, anthocyanins, hexanal, hexanol, linseed oil, and essential oils of thyme and citronella, biopolymers such as chitosan, xanthan gum, and cellulose nanoparticles, salts or ions such as CaCl₂, NaCl, Fe (IIII) ions, enzymes, and proteins of microbiological origin, as well as microorganisms or extracts thereof, all in food grade; additionally, disinfectant solutions for fruits and plants, components with antifungal activity such as thiabendazole, peracetic acid, or others allowed for post-harvest use in fruits with inedible peel, as well as components with aseptic activity against viruses, such as hydrogen peroxide, glutaraldehyde, peracetic acid, acetic acid, and salicylic acid.

14. The composite material (A) for a multifunctional coating product for plant structures according to claim 13, characterized in that the nutritional agents are selected from the group consisting of B-complex vitamins and vitamin D, fatty acids such as omega 3 and omega 6, ferric complexes, potassium, magnesium, inulin, and Lactobacillus.

15. A multifunctional coating (B) in dispersion for coating plant structures characterized in that it comprises a composite material (A) for a multifunctional coating product for plant structures comprising an in situ chemically modified polysaccharide, a plasticizing agent, a surfactant agent, and a solvent.

16. The multifunctional coating (B) in dispersion according to claim 16, characterized in that the polysaccharide is in a ratio of 20% to 40% w/w, the plasticizing agent is in a ratio of 2 to 23% w/w, and the surfactant agent is in a ratio of 2 to 23% w/w.

17. The multifunctional coating (B) in dispersion according to claim 16, characterized in that it further comprises complementary active agents which are between 0% and 12% and complementary nutritional agents which are between 0% and 12%.

18. The multifunctional coating (B) in dispersion for plant structures according to claim 18, characterized in that the complementary active agents are in a ratio of 0 mg/ml to 1 mg/ml and wherein the complementary active agents are selected from oleic acid, linoleic acid, sucrose monolaurate, sucrose monodecanoate, glyceryl monostearate, tannic acid, gallic acid, anthocyanins, hexanal, hexanol, linseed oil, essential oils of thyme and citronella, ascorbic acid, gallic acid, CaCl₂, NaCl, Fe (IIII) ions, enzymes and proteins of microbiological origin, as well as microorganisms or extracts thereof, all in food grade, and disinfectant solutions for fruits and plants.

19. The multifunctional coating (B) in dispersion for plant structures according to claim 18, characterized in that the complementary nutritional agents are in a ratio of 0 mg/ml to 1 mg/ml and wherein the complementary nutritional agents are selected from the group consisting of B-complex vitamins, vitamin D, omega 6, iron complexes, potassium, magnesium, inulin, and Lactobacillus

20. The multifunctional coating (B) in dispersion for plant structures according to claim 16, characterized in that the composite material (A) is in a ratio of 0.5 mg/ml to 50 mg/ml.

21. The multifunctional coating (B) in dispersion for plant structures according to claim 16, characterized in that the solvent is a solution of aqueous solvents or water-in-oil emulsions, wherein the solvent is selected from the group consisting of alcohol, oils, surfactants, and mixtures thereof.

22. The multifunctional solution coating for plant structures according to claim 22, characterized in that the solvent contains between 80% and 100% water, and the complement is the mixture of alcohol, oils, and surfactants.

23. The method for the preparation or manufacture of the composite material (A) for a multifunctional coating product for plant structures characterized in that it comprises the following steps:
a) Preparing a solution of plasticizing agent in water, wherein the plasticizing agent is at a concentration of 5% to 25% w/w with respect to the polysaccharide in solid state, wherein the solution is stirred at a speed between 300 and 500 rpm at a temperature between 18° C. and 30° C.;
b) Adding to the solution prepared in step a) a chemical modifying agent at a concentration of 5% to 30% w/w with respect to the polysaccharide, maintaining the stirring between 300 and 500 rpm and the temperature between 25° C. and 30° C.;
c) Adding to the solution prepared in step b) the polysaccharide at a mass aggregation rate of 5 to 10 g/s until reaching a concentration between 1% to 10% w/v and raising the temperature between 30° C. and 50° C.;
d) Solubilizing and hydrolyzing the polysaccharide in situ by increasing the temperature at a rate of 0.5 to 5° C./min until reaching a temperature between 80° C. and 90° C.;
e) Maintaining the final temperature and stirring constant between 300 and 500 rpm;
f) Condensing by reducing the temperature at a constant rate until reaching room temperature (18° C.-30° C.); and
g) Drying the product obtained in step f) by spray-drying with drying air at a temperature of 110° C.-200° C. and a flow rate of 100-1000 L/h or forced convection with reduced humidity of warm air at room temperature (18° C.-60° C.).

24. The method for the preparation or manufacture of a solid composition (A) for a multifunctional coating product for plant structures according to claim 24, characterized in that in step b) the chemical modifying agent is selected from the group consisting of citric acid, isocitric acid, aconitic acid, propan-1,2,3-tricarboxylic acid, and trimesic acid, all in food grade.

25. The method for the preparation or manufacture of a solid composition (A) for a multifunctional coating product for plant structures according to claim 24, characterized in that in step b) the chemical modifying agent is food grade citric acid.

26. The method for the preparation or manufacture of a solid composition (A) for a multifunctional coating product for plant structures according to claim 24, characterized in that it further comprises a step of adding a surfactant by permanent drip at a temperature between 18° C. and 80° C. with stirring between 500 and 1000 rpm before or after step f).

27. The method for the preparation or manufacture of a solid composition (A) for a multifunctional coating product for plant structures according to claim 24, characterized in that after drying, particle size is reduced by grinding, breaking, or cutting.

28. The method for the preparation or manufacture of a solid composition (A) for a multifunctional coating product for plant structures according to claim 24, characterized in that it further comprises adding active agents and nutritional agents at any step other than step c).

29. The method for the preparation or manufacture of a solid composition (A) for a multifunctional coating product for plant structures according to claim 24, characterized in that the polysaccharide is selected from the group consisting of starches, celluloses, and other heteropolysaccharides, all in food grade.

30. The method for the preparation or manufacture of a solid composition (A) for a multifunctional coating product for plant structures according to claim 24, characterized in that the polysaccharide is selected from starches, celluloses selected from the group consisting of derivatives thereof such as cellulose acetate, microcrystalline cellulose, hydroxypropylcellulose, and hydroxyethylcellulose, and the other heteropolysaccharides are selected from kefiran and hemicellulose, all in food grade and commercially available.

31. The method for the preparation or manufacture of a composite material (A) for a multifunctional coating product for plant structures according to claim 24, characterized in that the plasticizing agent is selected from polyols (polyalcohols), surfactants, polycarboxylic acids, and water.

32. The method for the preparation or manufacture of a composite material (A) for a multifunctional coating product for plant structures according to claim 24, characterized in that the plasticizing agent is selected from polyols from the group consisting of glycerol/glycerin, propylene glycol, and sorbitol, all in food grade.

33. The method for the preparation or manufacture of a composite material (A) for a multifunctional coating product for plant structures according to claim 24, characterized in that the surfactant agent is selected from polysorbates, proteins, sucrose esters of fatty acid, and monoacylglycerides.

34. The method for the preparation or manufacture of a composite material (A) for a multifunctional coating product for plant structures according to claim 34, characterized in that the surfactant agent is polyoxyethylene (20) sorbitan monolaurate (Tween 20).

35. The method for the preparation or manufacture of a composite material (A) for a multifunctional coating product for plant structures according to claim 24, characterized in that the active agents are selected from oleic acid, linoleic acid, sucrose monolaurate, sucrose monodecanoate, glyceryl monostearate, tannic acid, gallic acid, anthocyanins, hexanal, hexanol, linseed oil, essential oils of thyme and citronella, ascorbic acid, gallic acid, CaCl₂, NaCl, Fe (IIII) ions and microorganisms, all in food grade, and disinfectant solutions for fruits and plants.

36. The method for the preparation or manufacture of a composite material (A) for a multifunctional coating product for plant structures according to claim 24, characterized in that the nutritional agents are selected from the group consisting of B-complex vitamins and vitamin D, fatty acids such as omega 3 and omega 6, ferric complexes, potassium, magnesium, inulin, and Lactobacillus.

37. A method for obtaining a multifunctional coating (B) in dispersion for plant structures from a solid composition (A) for a multifunctional coating product in solution for plant structures characterized in that it comprises the steps of:
i. Forming a paste of the solid composition (A) by wetting and machining by adding water or solvent in a ratio of solid composition (A): solvent or water 1:1 to 5:1;
ii. Adding the paste obtained in step i) to the solvent or water up to a concentration of solid composition (A) between 0.5 g to 50 g per liter of solvent and stirring until obtaining a homogeneous solution;
iii. Adding optional active and nutritional agents.

38. The method for obtaining a multifunctional coating (B) in dispersion for plant structures from a solid composition (A) for a multifunctional coating product in solution for plant structures according to claim 38, characterized in that it comprises an additional step of adding active and nutritional agents before, during, or after step ii).

39. A method of depositing a multifunctional coating in dispersion for plant structures from a solid composition (A) characterized in that it comprises the steps of:
A. Contacting the soluble coating solution with the surface of the plant or fruit;
B. Drying the plant or fruit.

40. The method of depositing a multifunctional coating for plant structures from a composite material (A) according to claim 40, characterized in that step A is selected from solution dipping, brushing, spraying, or bathing.

41. The method of depositing a multifunctional coating for plant structures from a composite material (A) according to claim 40, characterized in that step B is selected from an operation of exposure to ambient conditions, convective drying by dry air (or low humidity), at room temperature or above, convective oven at low temperatures, or vacuum or low humidity chambers with air extraction.
